(19) United States
(12) Patent Application Publication
(10) Pub. No.: US 2011/0027777 A1
(43) Pub. Date: Feb. 3, 2011

(54) METHOD FOR PERFORMING PROGNOSIS FOR HIGH-RISK BREAST CANCER PATIENTS USING TOP2A GENE ABERRATIONS

(75) Inventors: Jan Trøst Jørgensen, Fredensborg (DK); Bent Ejlertsen, Brenshej (DK); Henning Mouridsen, Holte (DK); Kirsten Vang Nielsen, Bronshoj (DK)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: DAKO DENMARK, Glostrup (DK)

(21) Appl. No.: 12/293,631

(22) PCT Filed: Mar. 30, 2007
(86) PCT No.: PCT/DK2007/000160

§ 371 Date: Oct. 21, 2010

Related U.S. Application Data
(60) Provisional application No. 60/787,750, filed on Apr. 1, 2006.

(51) Int. Cl. C12Q 1/68

(52) U.S. Cl. 435/6

(57) ABSTRACT

A first exemplary method for performing a prognosis for a breast cancer patient, comprises the steps of determining the status of an aberration of the TOP2A gene in a tissue sample taken from the patient; and estimating the probability of either recurrence-free survival or of overall survival of the patient at a later time based upon a pre determined Hazard Ratio corresponding to the determined status. A second such exemplary method comprises the steps of determining the status of an aberration of the TOP2A gene in a tissue sample taken from the patient; and estimating the probability of either recurrence-free survival or of overall survival of the patient at a later time based upon a pre-determined Kaplan-Meier plot corresponding to the determined status.

### FIELD OF THE INVENTION

The present invention relates to prognoses for breast cancer patients. More particularly, the present invention relates to methods for performing such prognoses by determining the status (presence or absence and, if present, the type—amplification or deletion) of TOP2A gene aberrations.

### BACKGROUND OF THE INVENTION

The TOP2A gene is located on chromosome 17q21, in the same amplicon as HER2, where it codes for the enzyme topoisomerase IIα [see Järvinen TAH, Tanner M, Bärlund M, Borg Å, Isola J. “Characterization of Topoisomerase IIa Gene Amplification and Deletion in Breast Cancer.” Genes Chromosomes Cancer 1999; 26:142-150]. This enzyme is involved in the regulation of DNA topology and is important for the integrity of the genetic material during transcription, replication and recombination processes. During these processes topoisomerase IIα catalyzes the breakage and reunion of double stranded DNA [Osheroff N. “Biochemical basis for the interactions of type I and type II topoisomerases with DNA.” Pharmacol Ther 1989; 41(1-2):223-41; Roca J. “The mechanisms of DNA topoisomerases.” (review) Trends Biochem Sci 1995; 20(4):156-60; Wang J C. “Cellular roles of DNA topoisomerases: a molecular perspective.” Nat Rev Mol Cell Biol 2002; 3(6):430-40]. The expression of the topoisomerase IIα is cell cycle dependent with markedly higher levels in exponentially growing than in quiescent cell lines [Lynch B J, Guinee D G, Jr., Holden J A. “Human DNA topoisomerase II-alpha: a new marker of cell proliferation in invasive breast cancer.” Hum Pathol 1997; 28(10):1180-8; Hsiang Y H, Wu H Y, Liu L F. “Proliferation-dependent regulation of DNA topoisomerase II in cultured human cells.” Cancer Res 1988; 48(11):3230-5]. It has been shown that the amount of the enzyme correlates with cell proliferation [Heck MM, Earnshaw W C. “Topoisomerase II: A specific marker for cell proliferation.” J Cell Biol 1986; 103(6 Pt 2):2569-81].

The predominant genetic mechanism for oncogene activation is through amplification of genes that leads to protein over-expression and provides the tumor with selective growth advantages [Callagy G, Pharoah P, Chin S F, Sangan T, Daigo Y, Jackson L, et al. “Identification and validation of prognostic markers in breast cancer with the complementary use of array-CGH and tissue microarrays.” J Pathol 2005; 205(3):388-96]. Amplification of the TOP2A gene has been reported in 7-14% of patients with breast cancers and deletions with a similar frequency [Callagy et al, op cit.; Olsen K E, Knudsen H, Rasmussen B B, Balslev E, Knoop A, Ejlertsen B, et al. “Amplification of HER2 and TOP2A and deletion of TOP2A genes in breast cancer investigated by new FISH probes.” Acta Oncol 2004; 43(1):35-42; Harris L, Dressler L, Cowan D, Berry D, Cirrincione C, Broadwater G, et al. “The role of HER-2+Topo IIa Amplification in predicting benefit form CAF dose escalation CALGB 8541.” ASCO Annual Meeting 2004, Abstract no. 9505] In comparison, the HER2 oncogene is amplified in 20-30% of the breast cancer patients [Hayes D F, Thor A D. “c-erbB-2 in breast cancer: development of a clinically useful marker.” Semin Oncol 2002; 29(3):231-45].

Topoisomerase IIα is the pharmacological target of anthracyclines [Tewey K M, Rowe T C, Yang L, Halligan B D, Liu L F. “Adriamycin-induced DNA damage mediated by mammalian DNA topoisomerase II.” Science 1984; 226(4673):466-8; Hortobagyi G N. “Anthracyclines in the treatment of cancer. An overview.” Drugs 1997; 54 Suppl 4:1-7] and several studies have shown that TOP2A gene aberrations, especially amplification, are predictive to the response to anthracycline based chemotherapy in patients with breast cancer [Harris et al., op cit; Di Leo A, Gancberg D, Larsimont D, Tanner M, Jarvinen T, Rouas G, et al. “HER-2 amplification and topoisomerase IIalpha gene aberrations as predictive markers in node-positive breast cancer patients randomly treated either with an anthracycline-based therapy or with cyclophosphamide, methotrexate, and 5-fluorouracil.” Clin Cancer Res 2002; 8(5):1107-16; Park K, Kim J, Lim S, Han S. “Topoisomerase II-alpha (topoII) and HER2 amplification in breast cancers and response to preoperative doxorubicin chemotherapy.” Eur J Cancer 2003; 39(5):631-4; Press M F, Mass R D, Zhou J Y, Sullivan-Halley J, Villalobos I E, Lieberman G, et al. “Association of topoisomerase II-alpha (TOP2A) gene amplification with responsiveness to anthracycline-containing chemotherapy among women with metastatic breast cancer entered in the Herceptin H0648g pivotal clinical trial.” In: ASCO Annual Meeting; 2005; 2005. Abstract No. 9543; Tanner M M, Isola J J, Wiklund T, Erikstein B, Kellokumpu-Lehtinen P, Malmstrom P, et al. “Topoisomerase IIa gene amplification predicts favorable outcome of tailored and dose-escalated anthracyclin-based adjuvant chemotherapy in HER-2 positive breast cancer. Results from the randomized trial SBG 9401.” In: ASCO Annual Meeting; 2005; 2005. Abstract No. 9518; Knoop A S, Knudsen H, Balslev E, Rasmussen B B, Overgaard J, Nielsen K V, et al. “Retrospective analysis of topoisomerase IIa amplifications and deletions as predictive markers in primary breast cancer patients randomly assigned to cyclophosphamide, methotrexate, and fluorouracil or cyclophosphamide, epirubicin, and fluorouracil: Danish Breast Cancer Cooperative Group. J Clin Oncol 2005; 23(30):7483-90]. Fewer data are available with respect to patients with TOP2A deletions but a trend towards a better treatment outcome for this group of patients have been observed as well [Harris et al., op cit.; Knoop et al., op cit.] In contrast to the predictive properties of TOP2A gene aberrations, very little attention has been given to the prognostic value. So far one study has been published dealing with the subject and only in relation to TOP2A amplifications [Callagy et al., op cit.].

Recently, Callagy et al. [op cit.] published a study where they used the Fluorescence In-Situ Hybridization (FISH) technology on a tissue microarray in order to identify molecular markers that could be used to classify breast cancers into different prognostic groups. In that study, TOP2A and HER2 amplifications were shown to have a significant prognostic association with an adverse outcome of the disease. In the same study, topoisomerase IIα was measured using Immunohistochemistry (IHC). A significant association between TOP2A amplification and topoisomerase IIα was found. Over-expression of topoisomerase IIα was present in 93% of the cases with amplification of TOP2A. However, the other way around, only 20% of cases with overexpression had amplification. Unfortunately, heretofore, no information has been provided on the prognostic value of TOP2A deletion.

Other studies have failed to show a similar correlation [Petit T, Wilt M, Velten M, Millon R, Rodier J F, Borel C, et al. “Comparative value of tumour grade, hormonal receptors, Ki-67, HER-2 and topoisomerase II alpha status as predictive markers in breast cancer patients treated with neoadjuvant anthracycline-based chemotherapy.” Eur J Cancer 2004; 40(2):205-11; Mueller R E, Parkes R K, Andrulis I, O'Malley F P. “Amplification of the TOP2A gene does not predict high levels of topoisomerase II alpha protein in human breast tumor samples.” Genes Chromosomes Cancer 2004; 39(4):288-97; Durbecq V, Desmed C, Paesmans M, Cardoso F, Di Leo A, Mano M, et al. “Correlation between topoisomerase-IIalpha gene amplification and protein expression in HER-2 amplified breast cancer.” Int J Oncol 2004; 25(5):1473-9].

The association between topoisomerase IIα over-expression and established prognostic factors have been investigated in several studies [Rudolph P, MacGrogan G, Bonichon F, Frahm S O, de Mascarel I, Trojani M, et al. “Prognostic significance of Ki-67 and topoisomerase IIalpha expression in infiltrating ductal carcinoma of the breast. A multivariate analysis of 863 cases.” Breast Cancer Res Treat 1999; 55(1):61-71; Hellemans P, van Dam P A, Geyskens M, van Oosterom A T, Buytaert P, Van Marck E. Immunohistochemical study of topoisomerase II-alpha expression in primary ductal carcinoma of the breast. J Clin Pathol 1995; 48(2):147-50; Rudolph P, Olsson H, Bonatz G, Ratjen V, Bolte H, Baldetorp B, et al. “Correlation between p53, c-erbB-2, and topoisomerase II alpha expression, DNA ploidy, hormonal receptor status and proliferation in 356 node-negative breast carcinomas: prognostic implications.” J Pathol 1999; 187(2):207-16; Depowski P L, Rosenthal S I, Brien T P, Stylos S, Johnson R L, Ross J S. “Topoisomerase IIalpha expression in breast cancer: correlation with outcome variables.” Mod Pathol 2000; 13(5):542-7; Kalogeraki A, leromonachou P, Kafousi M, Giannikaki E, Vrekoussis T, Zoras O, et al. “Topoisomerase II alpha expression in breast ductal invasive carcinomas and correlation with clinicopathological variables.” In Vivo 2005; 19(5):837-40]. Most of these studies have shown some kind of association between the overexpression of the enzyme and an adverse outcome of the disease. Using a polyclonal antiserum (Ki-S4) for staining of specimens from node-negative breast cancer patients, Rudolph, McGrogan et al. [op cit.] and Rudolph, Olsson et al. [op cit.] showed that topoisomerase IIα overexpression was an independent prognostic factor for survival.

### SUMMARY OF THE INVENTION

In order to extend the available methods for performing prognoses for breast cancer patients, beyond what is presently available in the art, novel methods for performing such prognoses are herein disclosed, wherein the prognoses are based upon the determined status of TOP2A gene aberrations (wherein the term “status” refers to the presence or absence of an aberration and, if an aberration is present, the type—amplification or deletion—of the aberration). Embodiments in accordance with the invention may comprise the steps of determining the status of an aberration of the TOP2A gene in a tissue sample taken from a patient; and estimating the probability of either recurrence-free survival or of overall survival of the patient at a later time based upon either a pre-determined Hazard Ratio or a pre-determined Kaplan-Meier estimator plot of recurrence-free survival (RFS) or of overall survival (OS) corresponding to the determined status.

### DESCRIPTION OF THE DRAWINGS

FIG. 1 shows a chart of patient disposition in an exemplary study conducted according to a method in accordance with the present invention

FIG. 2 presents a bar graph showing the associations between TOP2A status and age at surgery for patients (number of patients, N=773) studied in the exemplary study of FIG. 1; Abbreviations: Del-deletion; Amp-amplification; Nor-normal

FIG. 3 presents a bar graph showing the associations between TOP2A status and tumor size for patients (number of patients, N=773) studied in the exemplary study of FIG. 1; Abbreviations: Del-deletion; Amp-amplification; Nor-normal

FIG. 4 presents a bar graph showing the associations between TOP2A status and number of positive lymph nodes for patients (number of patients, N=773) studied in the exemplary study of FIG. 1; Abbreviations: Del-deletion; Amp-amplification; Nornormal

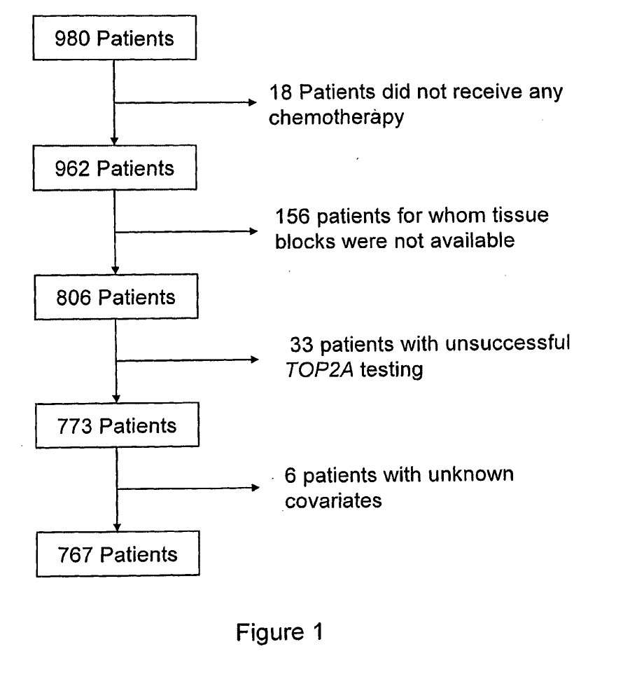

FIG. 5: (a) shows a Kaplan-Meier estimator plot of recurrence-free survival (RFS) of patients studied in the exemplary study of FIG. 1; number of patients (N=767) is plotted versus determined TOP2A aberrations.
(b) shows a Kaplan-Meier estimator plot of overall survival (OS) of patients studied in the exemplary study of FIG. 1; number of patients (N=767) is plotted versus various determined TOP2A aberrations

FIG. 6: (a) shows a Kaplan-Meier estimator plot of recurrence-free survival (RFS) of patients studied in the exemplary study of FIG. 1; number of patients) (N=767) is plotted versus determined HER2 status
(b) shows a Kaplan-Meier estimator plot of overall survival (OS) of patients studied in the exemplary study of FIG. 1; number of patients (N=767) is plotted versus determined HER2 status

FIG. 7 shows a Kaplan-Meier estimator plot of recurrence-free survival (RFS) of patients treated with CMF (Cyclophosphamide Methotrexate 5-Fluorouracil); number of patients (N=418) is plotted versus determined HER2 status

FIG. 8 shows a Kaplan-Meier estimator plot of recurrence-free survival (RFS) of patients treated with CEF (Cyclophosphamide Epirubicin 5-Fluorouracil); number of patients (N=349) is plotted versus determined HER2 status

FIG. 9 shows a Kaplan-Meier estimator plot of overall survival (OS) of patients treated with CMF (Cyclophosphamide Methotrexate 5-Fluorouracil); number of patients (N=418) is plotted versus determined HER2 status Comparison of OS by TOP2A when treated with CMF (N=418)

FIG. 10 shows a Kaplan-Meier estimator plot of overall survival (OS) of patients treated with CEF (Cyclophosphamide Epirubicin 5-Fluorouracil); number of patients (N=349) is plotted versus determined HER2 status Comparison of OS by TOP2A when

### DETAILED DESCRIPTION OF THE INVENTION

The present invention provides new methods for prognosis of breast cancer in a patient, wherein said prognosis is based on determining the status of the TOP2A gene aberrations.

By term “prognosis” is meant a statement of what is judged likely to happen in the future, especially in connection with a particular situation, more particularly a judgment of the likely or expected development of a disease or of the chances of getting better.

By the term “gene aberration” is meant any change in the DNA sequence of a gene or a change in a sequence/region related to a gene, e.g. a regulatory chromosomal region of the gene. The term “gene” in the present context means a locatable region of genomic sequence, corresponding to a unit of inheritance, which is associated with regulatory regions, transcribed regions and/or other functional sequence regions. Preferable gene aberrations may be selected but not limited to amplifications, duplications and/or deletions of the whole DNA sequence of a gene, fragments/parts of the gene sequence and/or gene-related sequences in the subject genome or parts of said sequences.

A sequence/gene/region, where the status of an aberration to be determined, is termed herein as “target sequence/gene/region” or “sequence/gene/region of interest”.

The term “subject” in the present context means any mammal including human having or suspected of having a disease. The term “subject” is herein used interchangeably with the term “patient”.

Thus, a first aspect of the invention relates to performing prognosis for a breast cancer patient by determining the status of an aberration of the TOP2A gene. The status of an aberration of the gene is determined by performing gene analysis in a sample, such as a tissue or cell sample, taken from said cancer patient. The term “status of an aberration” means the presence or absence of an aberration, and if present, the type of aberration. When an aberration is absent the gene is herein referred as normal. For example, the absence of amplification or deletion of a gene is reflected by the presence of the gene in a normal number of copies.

To estimate the status of an aberration of a gene, reference genomic sequences may be used. By the term “reference sequence” is meant a sequence which is not identical with the gene/sequence/region of interest. By applying a reference sequence located on the same chromosome as a gene of interest, the specific ploidy level of the given chromosome is decisive of whether a genomic target sequence (a sequence, the status of aberration of which is to be determined) will be found amplified, deleted or normal. Examples of preferable reference sequences are discussed below.

Determining the status of an aberration of the gene of interest is preferably done by using gene analysis, wherein the term “gene analysis” means any analysis that may be suitable for analyzing genes, e.g. in situ hybridization, RT-PCR.

To perform gene analysis various probes may be used. “Probe” as used herein means any molecule or composition of molecules that may bind to the region(s)/sequence(s) related to the gene to be detected or visualized.

The invention in different embodiments may relate to different types of probes, e.g.
specific probe, which means any probe capable of binding specifically to regions to be detected, i.e. a genomic sequence related to the gene which status of aberration is to be determined, a sequence of the gene product, such as protein or RNA molecule;
blocking probe, which means any probe capable of blocking, suppressing or preventing the interaction of a region to be detected with other probes or molecules,

The origin of the probes may in different embodiments also be different, e.g.
nucleic acid probe, which include any molecule of a naturally occurring nucleobase sequence-containing oligomer, polymer, or polymer segment, having a backbone formed solely from nucleotides, or analogs thereof; “nucleotide” as used herein, means any of several compounds that consist of a ribose or deoxyribose sugar joined to a purine or pyrimidine base and to a phosphate group. Nucleotides are the basic structural subunits of nucleic acids. Examples of nucleic acid probes may probes comprising sequences of DNA and/or RNA.
nucleic acid analog probe, which means any molecule that is not a naturally occurring nucleic acid molecule or is composed of at least one modified nucleotide, or subunit derived directly from a modification of a nucleotide. An example of nucleic acid analog probes may be probes comprising sequences of PNA, wherein “PNA” is the abbreviation of peptide nucleic acid;
protein probes made from whole protein molecules such as e.g. antibodies, receptors, ligands, growth factors, DNA binding proteins or any other protein that may bind a region of interest, or
peptide probes comprising shorter peptide sequences derived from the above proteins or peptide probes comprising synthetic peptide sequences comprising natural and/or unnatural amino acids residues.

Nucleic acid probes of the invention may be made up of naturally occurring nucleic acid molecules, such as oligodeoxynucleic acids (e.g. DNA), oligoribonucleic acids (e.g. RNA, mRNA, siRNA), and fragments thereof. Nucleic acid analogue probes may bind to the same region of interest as the nucleic acid probes and may be made from modified naturally occurring nucleic acid molecules or may be synthetic molecules. Non-limiting examples of a modified naturally occurring molecule may be Locked Nucleic Acid (LNA) or synthetic molecules which are polyamide based such as e.g. Peptide Nucleic Acid (PNA), or other nucleic acid analogs or nucleic acid mimics.

The probes may have any length suitable for detecting the target region, e.g. TOP2A gene, a reference sequence of the subject genome, such as a centromeric region. Usually a probe is made up of smaller fragments of varying sizes (e.g. about 50 by to about 500 by each) such that the probe will in total span about 30 kb to about 2 Mb. The probe will usually comprise both unique fragments as well as repeated fragments. If such repeated fragments are undesirable in the probe sequence, they can be removed or blocked, for example by using blocking probes.

Nucleic acid analogue probes, like PNA probes, are usually shorter, well defined probes, typically comprising from about 10 to 25 nucleobases. A PNA probe is usually composed of several individual probes, each having 10 to 25 nucleobase units.

Nucleic acid probes, nucleic acid analogue probes and protein probes may be employed in separate analyses or in combination in the same analysis. A non-limiting examples could be the employment of one-two nucleic acid probes for detection of the sequence of interest and either a nucleic acid, nucleic acid analogue probe or protein probe for detection of the reference sequence or product of the reference gene, such as a protein or RNA.

Probes may be, and in some preferred embodiments are, labeled.

Labeling of the probes may be done using different well-known in the art methods, e.g. by means of enzymatic or chemical processes. Any labeling method known to those in the art can be used for labeling probes for the purposes of this invention.

Probes may bind to a sequence of the gene of interest, or another reference sequence, and hybridize under stringent conditions. Those of ordinary skill in the art of hybridization will recognize that factors commonly used to impose or control stringency of hybridization include formamide concentration (or other chemical denaturant reagent), salt concentration (i.e., ionic strength), hybridization temperature, detergent concentration, pH and the presence or absence of chaotropes. Optimal stringency for a probe/marker sequence combination is often found by the well-known technique of fixing several of the aforementioned stringency factors and then determining the effect of varying a single stringency factor. The same stringency factors can be modulated to thereby control the stringency of hybridization of a PNA to a nucleic acid, except that the hybridization of a PNA is fairly independent of ionic strength. Optimal stringency for an assay may be experimentally determined by examination of each stringency factor until the desired degree of discrimination is achieved. Generally, the more closely related the background causing nucleic acid contaminates are to the target sequence, the more carefully stringency must be controlled. Suitable hybridization conditions will thus comprise conditions under which the desired degree of discrimination is achieved such that an assay generates an accurate (within the tolerance desired for the assay) and reproducible result. Nevertheless, aided by no more than routine experimentation and the disclosure provided herein, those of skill in the art will easily be able to determine suitable hybridization conditions for performing assays utilizing the methods and compositions described herein.

Non-limiting examples of stringent conditions are described in the experimental procedure below and further non-limiting examples may be found in chapter 11 in Peptide Nucleic Acids, Protocols and Applications, Second Ed. Editor Peter E Nielsen, Horizon Scientific Press, 2003.

The probe binding to a reference sequence may be targeted against the centromeric region of the chromosome where the gene of interest, i.e the TOP2A gene, is located. By applying the reference on the same chromosome, the specific ploidy level of the given chromosome is decisive of whether the genomic probe will be found amplified, deleted or normal. Both nucleic acid probes, nucleic acid analogue probes as well as protein probes may be employed. In spite of the great homology in the centromeric DNA of humans, clones have been identified and constructed, containing human chromosome specific centromeres for use in FISH assays as the reference sequences. Probe length may be dramatically reduced without reduction of the signal intensity when probes targeted against centromeric repeat sequences are used. The advantage of using centromeric reference probes is that they do not contribute to background staining as they do not contain SINEs and LINEs.

Centromeric regions, e.g. of chromosome 17 where the TOP2A gene is located, have been found to be specifically identified by FISH probes derived from clone sequences that can be used directly as reference probes. However, synthetic peptide nucleic acid (PNA) probes may be chosen for centromere detection in FISH, because of their DNA specificity and higher signal intensity, with a reduction of unspecific background. A PNA is a synthetic oligonucleotide where the backbone mimics a peptide instead of the deoxyribose phosphodiester backbone of DNA. For PNA construction a sequence of about 10-25 bases is useful. Alternatively a locus specific probe (LSP) may be used as reference. This should preferably be placed on the opposite chromosome arm than the gene of interest, to eliminate incorrect probe to reference ratio if whole arm deletions occurs. The reference probe should not be placed in a region that has any relation to genome aberrations in cancer.

A number of gene analyses are known where the probes described above may successfully be used. Many of these analyses have already become a part of laboratory routine. The precise analysis used in the method according to the invention should be carefully selected according to the nature of sequences of interest and the probes.

Fluorescence in-situ hybridization (FISH) is an important tool for determining the number, size and/or location of specific DNA sequences in cells and may be applied in the method of the invention. Typically, the hybridization reaction fluorescently stains the sequences so that their location, size and/or number can be determined using fluorescence microscopy, flow cytometry or other suitable instrumentation. DNA sequences ranging from whole genomes down to several kilobases can be studied using current hybridization techniques in combination with commercially available instrumentation. In Comparative Genomic Hybridization (CGH) whole genomes are stained and compared to normal reference genomes for the detection of regions with aberrant copy number. In the m-FISH technique (multi color FISH) each separate normal chromosome is stained by a separate color (Eils et al, Cytogenetics Cell Genet 82: 160-71 (1998)). When used on abnormal material, the probes will stain the aberrant chromosomes thereby deducing the normal chromosomes from which they are derived (Macville M et al., Histochem Cell Biol. 108: 299-305 (1997)). FISH-based staining is sufficiently distinct such that the hybridization signals can be seen both in metaphase spreads and in interphase nuclei. Single and multicolor FISH, using nucleic acid probes, have been applied to different clinical applications generally known as molecular cytogenetics, including prenatal diagnosis, leukemia diagnosis, and tumor cytogenetics. Other gene analysis methods of the application may be RT-PCR and CISH (Chromogenic In Situ Hybridization). In particular, a combination of FISH and CISH may be used, e.g. by labeling the probe with a fluorescent label or chromogen label, and subsequently converting the FISH signal into a CISH signal or visa versa. Alternatively, a probe can be labeled with both a fluorescent and a chromogen label so as to enable separate detection of the FISH signal or the CISH signal.

A gene analysis preferably performed using a tissue sample, e.g. a biopsy sample. The simplest way to perform the analysis may be to cut the relevant number of sections from paraffin embedded tissue and hybridize a probe to each section. Alternatively frozen tissue can be used or imprints. Hybridization demands only standard conditions. For most probes an internal reference, such as e.g. a centromeric probe, preferably to be included. The gene probe and the reference probe should be labeled differently, e.g. with labels which generate different colors such as e.g. red and green, respectively. Non-limiting examples of such labels may be fluorescent labels Texas Red and Fluorescein. The blue DAPI color may be used for counterstaining to assist tissue localizetion and identification. Availability of control Hematoxylin-Eosin cut section may also be useful.

The status of an aberration of the gene may be measured as the actual number of copies of the sequence of interest present in the sample, e.g. number of copies of the gene, i.e. the TOP2A gene or the gene related sequence. The status of an aberration of the gene may also be determined as the actual amount of a gene product in the sample, e.g. total amount of the gene corresponding RNA or protein. The status of an aberration of the gene may also be reported as a ratio, where the amount of the sequence of interest is correlated to the amount of a reference sequence. In some embodiments it is preferred to use the latter evaluation. The level of an aberration of the gene is correlated to the condition of interest, i.e. a breast cancer, and may therefore be used for describing and/or predicting such conditions or diseases and development thereof. Sometimes the status of a gene aberration is referred to as cut-off values.

In a normal cell two copies of each of our genes are present. Theoretically, two signals derived form the probe bound to the complementary DNA strands should be visible. However, in some embodiments, in a sample prepared for performing gene analysis by in situ hybridization, due to cutting of sections from paraffin embedded tissue, whole nuclei will not be present. Therefore, a difference between theoretical and actual number of signals may be observed and cut-off values between normal and abnormal number of signals per cell will have to be determined empirically. Using a reference probe, two reference probe signals should be seen in a normal cell, and theoretically the ratio between signals from gene probe and reference probe should be 1 (one). However, due to technical, biological and statistical reasons this absolute value is determined as a range, e.g. such as a range between 0.8 and 2.0 in the case of HER2 FISH (package insert, Dako HER2 FISH pharmDx™ kit, code K5331). The FISH assay can be performed with and without one or more reference probes. Without a reference probe only signals in one color from the target gene probe are scored, and the cut-off value between normal and amplified gene sequence is 4-5, although the theoretical value is 2. Deletions cannot be scored in an assay without a reference probe or a reference sample.

A FISH assay may include one or more reference probes in addition to the gene probe, e.g. the TOP2A gene probe and centromere probe labeled differently, e.g. with different fluorescent labels. The gene copy number may then be calculated by using the reference probe. Signal from each gene copy and signal from the corresponding reference sequence are detected and the ratio is calculated. As already mentioned, the reference sequence is a measure of the ploidy level, thus it indicates the number of chromosome copies. The most accepted cut-off value of a normal gene copy number is indicated by a ratio between 0.8 and 2.0. Gene deletion is indicated by a ratio below 0.8, whereas gene amplification is indicated by a ratio above 2.0.

According to the invention, for the TOP2A gene a cut-off value between 0.8 and 2 is indicative of a normal gene copy number and is predictive of better recurrence-free survival or overall survival of a patient, whereas the presence of an aberration of the gene, reflected by a decreased (a cut-off value less than 0.8) or increased gene copy number (a cut-off value more than 2) is predictive of a worse prognosis, such as a worse recurrence-free survival or overall survival of a patient.

Prognostic value, of the determined status of an aberration of the TOP2A gene is illustrated herein by non-limiting examples and discussed further in detail in the section Examples.

In specific embodiments the invention relates

1. to a method for performing a prognosis for a breast cancer patient, comprising the steps of:
determining the status of an aberration of the TOP2A gene in a tissue sample taken from the patient; and
estimating the probability of either recurrence-free survival or overall survival of the patient at a later time based upon a pre-determined Hazard Ratio corresponding to the determined status;
   2. to a method for performing a prognosis for a breast cancer patient, comprising the steps of:
determining the status of an aberration of the TOP2A gene in a tissue sample taken from the patient; and
estimating the probability of either recurrence-free survival or of overall survival of the patient at a later time based upon a pre-determined Kaplan-Meier plot corresponding to the determined status.

In one embodiment the probability of either recurrence-free survival or overall survival of the patient at a later time may be determined based upon a pre-determined Hazard Ratio corresponding to the determined status. In another embodiment the probability of either recurrence-free survival or overall survival of the patient at a later time may also be determined based upon a pre-determined Kaplan-Meier plot corresponding to the determined status.

In some embodiments both recurrence-free survival and of overall survival of the patient at a later time may be determined based upon a pre-determined Hazard Ratio and Kaplan-Meier plot.

In one embodiment the pre-determined Hazard Ratio is calculated by performing steps comprising:
determining the status of aberrations of the TOP2A gene in a set of tissue samples taken from respective patients; and
performing subsequent follow-up studies of recurrence-free survival time or of overall survival time for the patients.

The term “determined status” in the present content is meant the status of a gene aberration determined in the sample.

In one embodiment the determined status corresponds to TOP2A amplification.

In another embodiment the determined status corresponds to TOP2A deletion.

In another embodiment the determined status corresponds to normal TOP2A.

As discussed above, determining the status of an aberration of the TOP2A gene may be performed by any gene analysis known in the art. In one preferred embodiment the step of the determining may include conducting a FISH analysis of the tissue sample, in another preferred embodiment it may include a CISH analysis.

In one embodiment analysis of the status of an aberration of the TOP2A gene by in situ hybridization may comprise a step of using a mixture of probes. The number of probes in the mixture is not limited and may comprise two or more different or identical probes. In one embodiment it may be a mixture of probes, wherein at least one probe is targeted at a portion of the gene and at least one another probe is targeted at a portion of the centomeric region of chromosome 17, wherein both probes are nucleic acid probes, such as DNA probes. In another embodiment the mixture of hybridization probes may comprise both nucleic acid probes and nucleic acid analog probes, preferably a mixture of DNA probes and PNA probes; preferably, the DNA probes are targeted at a portion of the TOP2A region and PNA probes are targeted at the centromeric region of chromosome 17. Preferably, the probes are labelled. Preferably the DNA probes are labelled differently from the PNA probes. The labels may be any labels, e.g. luminescent, fluorescent, chromogenic, enzymes labels or of any other origin. In some embodiments the mixture of probes may preferably include Texas Red-labelled DNA probes targeted at a portion of the TOP2A region and a mixture of fluorescein-labelled Peptide Nucleic Acid (PNA) probes targeted at the centromeric region of chromosome 17.

Analysis of samples using in situ hybridization and evaluation of the results may be performed by using manual or partially or fully automated protocols.

In one embodiment of the invention the method is further contemplating the use of image analysis systems.

Manual reading of the result of many samples is very time consuming. Therefore, it would be a great help to have access to automated systems. The reading of for example many fields of hybridization would be aided by fluorescence image analysis with high speed scanning facilities. MetaSystems is an example of a provider of an image analysis system that might be used.

All the above described embodiments are illustrated by working examples described below.

### EXAMPLES

### Example 1

Based on data from the Danish Breast Cancer Cooperative Group (DBCG) trial 89-D the objective of the following described retrospective analysis was to investigate the prognostic value of TOP2A aberrations, both amplification and deletion, and HER2 status in high-risk breast cancer patients, so as to generate useful statistical data that may be employed in the performance of prognoses methods according to the invention.

### Patients and Methods

The details of DBCG 89-D study have been published elsewhere in Knoop et al. [op cit.]. In brief, women diagnosed with primary invasive breast cancer were eligible for the study if they were I: premenopausal, node-negative and with grade 2 or 3 tumors 5-5 cm; II: premenopausal with receptor negative or unknown tumors >5 cm or with positive axillary lymph nodes or III: postmenopausal with receptor negative tumors >5 cm or with positive axillary lymph nodes. Following mastectomy or tumorectomy, the patients were randomized to chemotherapy regimes of either CMF (cyclophosphamide 600 mg/m², methotrexate 40 mg/m² and 5-fluorouracil 600 mg/m²) or CEF (cyclophosphamide 600 mg/m², epirubicin 60 mg/m² and 5-fluorouracil 600 mg/m²). In total, 980 patients where enrolled in the study. Among these, 18 patients never received chemotherapy and were excluded from the study (see FIG. 1). Radiotherapy was given against the residual breast following lumpectomy (48 Gy+boost 10 Gy) or chest wall following mastectomy if the tumour was >5 cm (48 Gy), and against regional nodes in node-positive disease (48 Gy). In all cases, 2 Gy were administered in 5 fractions per week. In the CMF and CEF groups, 206 (40.0%) and 173 (38.7%), respectively received radiotherapy. The DBCG 89-D study and the retrospective TOP2A study were conducted according to the Helsinki declaration and approved by the Danish Ethical Committees.

### Preparation of Tissue

Among the 962 patients who received chemotherapy, tissue blocks were available from 806 patients (see FIG. 1). Consecutive serial sections were cut at 4 μm from the available paraffin-embedded tumors for immunohistochemistry (IHC) and Fluorescence In Situ Hybridization (FISH) and stored cold until staining was performed. All analyses were performed at the Department of Pathology, Roskilde Hospital, Denmark.

### HER2 Immunohistochemistry

The sections were stained within 5 days from cutting using a Techmate immunostainer (Dako, Glostrup, Denmark) according to the manufacturer's procedure procedures for the HercepTest™ (Dako, Glostrup, Denmark). Positive controls as supplied with the kit were included as well as in house controls together with a negative control for each case. The results were scored 0, 1+, 2+, and 3+ as recommended for the HercepTest™.

### TOP2A and HER2 FISH

The TOP2A FISH pharmDx™ Kit and HER2 FISH pharmDx™ Kit (Dako, Glostrup, Denmark) were each used on separate tissue slides according to the manufacturer's procedure. The ready-to-use TOP2A FISH Probe Mix included with the FISH pharmDx™ Kit is based on a combination of PNA (peptide nucleic acid) [Nielsen P E, Egholm M, editors. Peptide Nucleic Acids: Protocols and Applications. Norfolk NR18 0EH, England: Horizon Scientific Press; 1999] and DNA technology. This Probe Mix consists of a mixture of Texas Red-labeled DNA probes covering a total of 227 kb of the TOP2A region, and a mixture of fluorescein-labeled PNA probes targeted at the centromeric region of chromosome 17. The specific hybridization to the two targets results in formation of a distinct red fluorescent signal at each TOP2A gene and a distinct green fluorescent signal at each centromeric region of chromosome17. To diminish background staining, the Probe Mix also contains unlabeled PNA blocking probes. The reagent is provided in liquid form in hybridization solution containing 45% formamide, 10% dextran sulphate, 300 mmol/L NaCl, 5 mmol/L phosphate, and blocking agent. The working procedure for the two FISH pharmDx™ kits are described in the publication by Olsen et al [op cit.]. Up to 60 gene signals (or the number closest to 60+) were counted in nuclei with identifiable boundaries. The ratio was calculated as the number of signals from the gene probes (HER2 and TOP2A respectively) divided by the number of signals for the centromere 17. Cases were scored as HER2 or TOP2A FISH amplified when the ratio was 2. A TOP2A deletion is considered present when the ratio was <0.8.

### HER2 Status

HER2 FISH and HER2 IHC were performed on all tumor specimens. HER2 (3+) positive tumors or HER2 (2+) positive tumors with HER2 amplification (ratio 2) were considered HER2 positive. HER2 (0 and 1+) or HER2 (2+) positive tumors with no HER2 amplification (ratio<2) were considered HER2 negative.

The examination of all the slides was done blinded, i.e. data concerning tumor size, malignancy grade, receptor status, number of positive lymph nodes, adjuvant therapy, clinical outcome, etc. were unknown to the examiner.

### Statistical Analysis

The primary outcome of interest was recurrence-free survival (RFS) calculated as the time from randomization until first loco-regional recurrence, distal recurrence, second malignancy or death, and overall survival (OS) calculated as the time from randomization until death. Follow-up time was quantified in terms of a Kaplan-Meier estimate of potential follow-up [Schemper M, Smith T L. “A note on quantifying follow-up in studies of failure time.” Control Clin Trials 1996; 17(4):343-6]. Association between TOP2Astatus and the classical prognostic variables and HER2-status was investigated using contingency tables and Chi-square-test. Survival curves were constructed according to the Kaplan-Meier product-limit method and compared using the log-rank test. Multivariate survival analysis was conducted using Cox proportional hazards models with backward selection (Per Kragh Andersen, Ørnulf Borgan, Richard D. Gill, Niels Keiding: Statistical Models Based on Counting Processes, Springer-Verlag (1992), VII.2)

The proportional hazard assumption was assessed graphically as well as by including a time-dependent component individually for each covariate. Hormone receptor-status and malignancy grade were found to violate the assumption of proportional hazards. This was taken into account by stratifying for the two variables.

Cox Proportional Hazards regression analysis was carried out separately within the three subgroups consisting of TOP2A amplified, TOP2A deleted and TOP2A normal patients—and the two subgroups consisting of HER2 positive and negative patients. The prognostic value of a given characteristic was quantified by the hazard ratio (HR). The overall significance of interaction terms with two or more degrees of freedom was assessed by a Wald test.

The issue of potential selection bias was addressed. The distribution of clinical and pathological variables values was given for each of the two groups (patients with tissue-block available vs. patients with no tissue-block available) and the hypotheses of no difference in baseline values between the groups were tested by χ2-test. The RFS and OS were compared between the groups with and without available tissue by a log-rank test.

All patient records were updated regarding disease status and death in Dec. 31, 2004 and therefore all censored patients were known to be alive on Jan. 1, 2005.

### Results

The patient disposition for the study is shown in FIG. 1. Tissue blocks were missing from 156 patients (16%) of 980 patients initially randomized. Using Kaplan-Meier plots and the log-rank test it was shown that there was no significant difference for RFS and OS depending on whether the tissue was available or not. For menopausal status, tumor size, number of positive lymph node, hormone receptor status and malignancy grade, a significant difference was found. Tissue blocks were more often available for patients with: higher age, more positive lymph nodes, larger tumor size and higher malignancy grade.

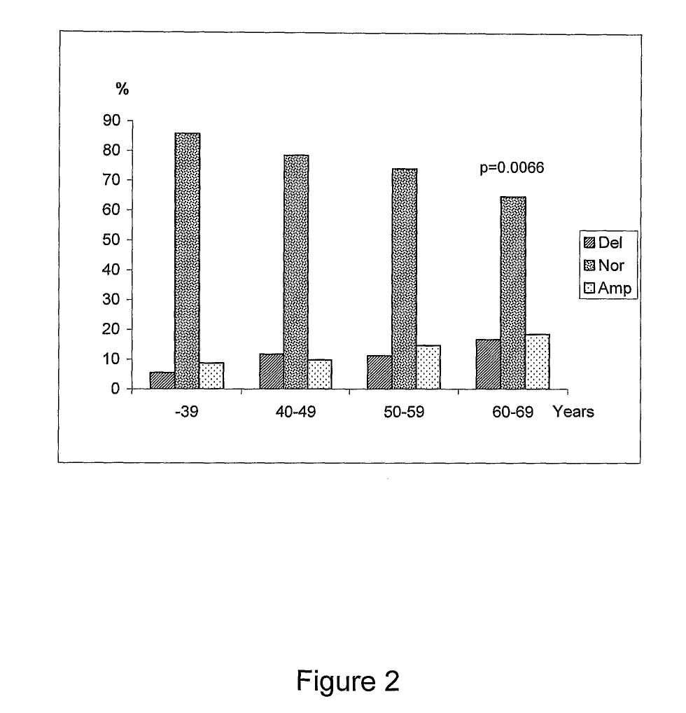

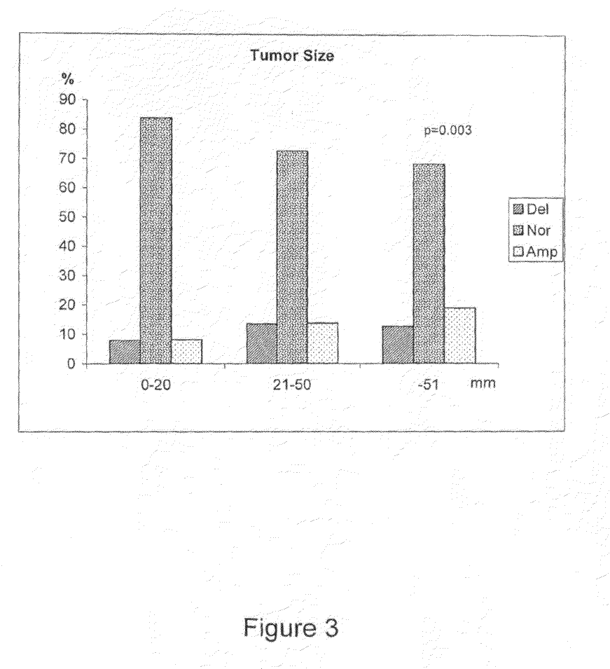

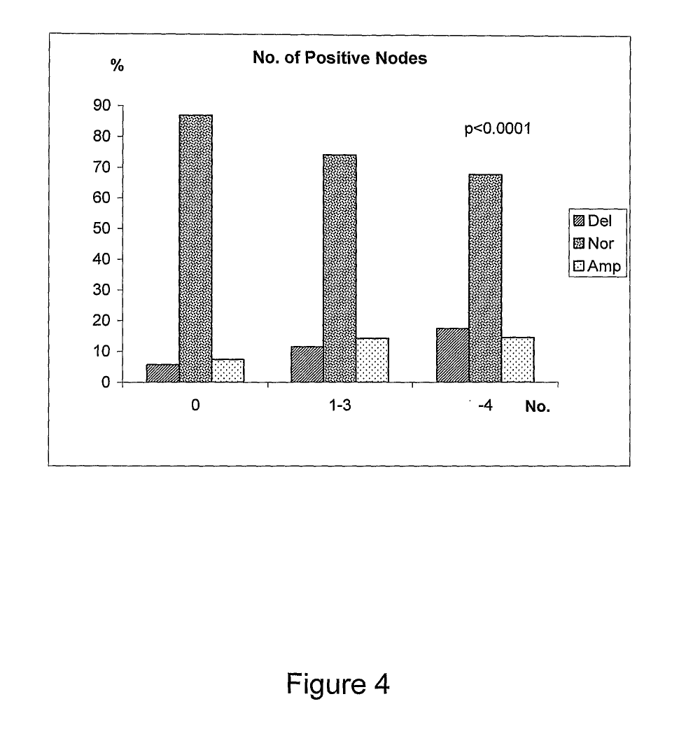

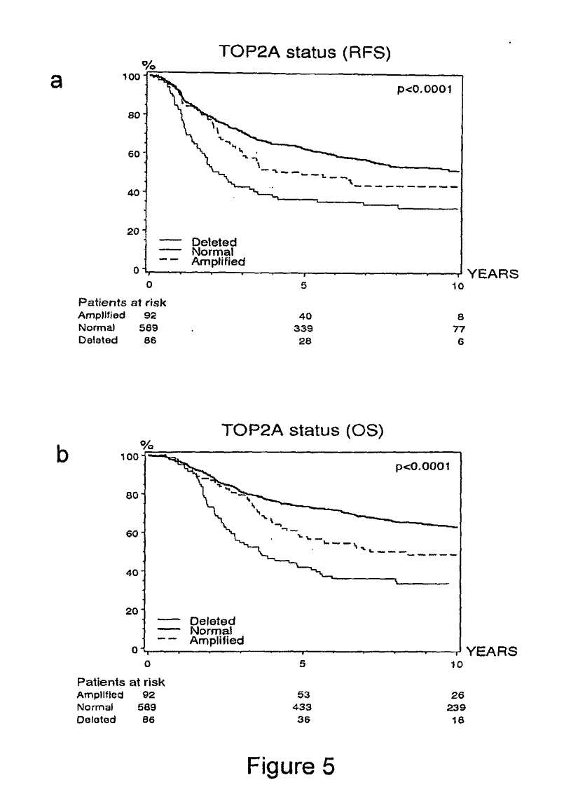

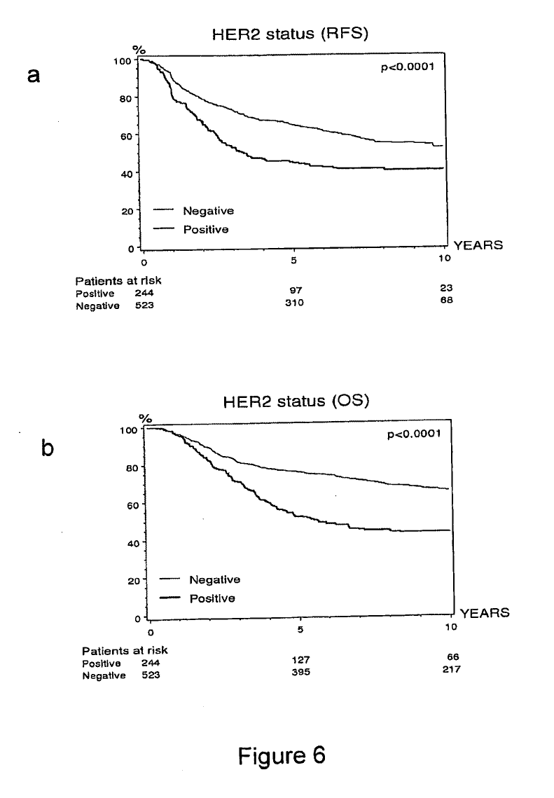

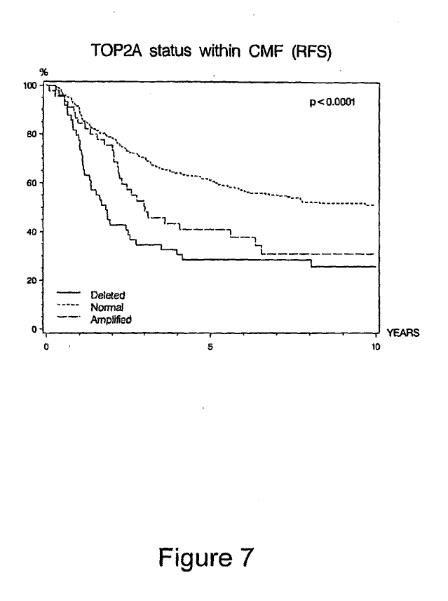

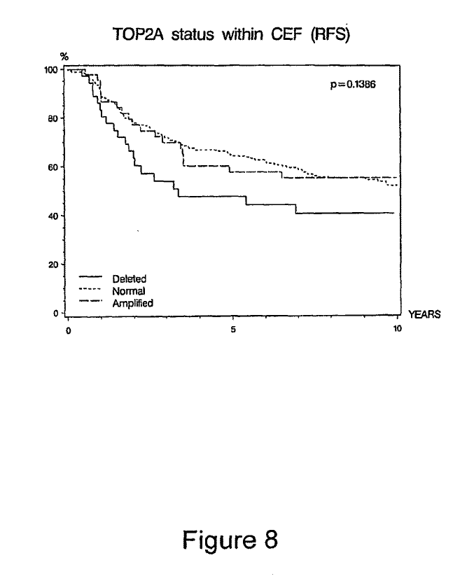

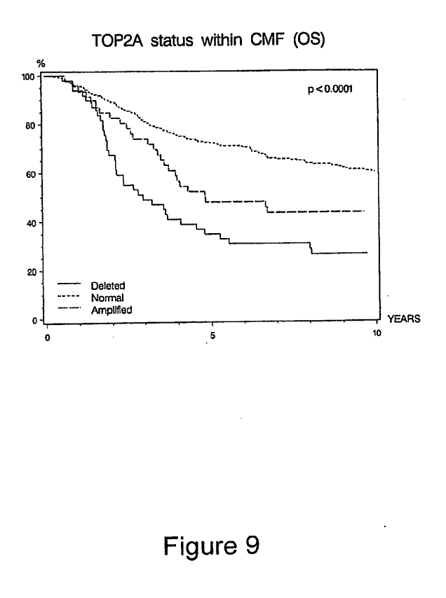

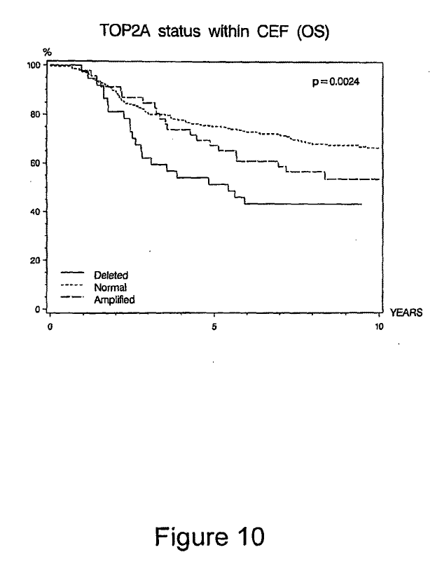

The TOP2A FISH test was successfully completed for 773 (96%) of 806 available tissue blocks. For the HER2 FISH and HER2 IHC tests, 805 out of 806 available tissue blocks were analyzed successfully. For the patients with TOP2A test results available the median potential follow-up time for RFS was 9.4 years and for OS11.1 years. Amplification of TOP2A was found in 92 (11.9%) and deletions in 87 (11.3%) of the 773 patients. The baseline patient's characteristics and the distribution of the TOP2A status in relation to clinical and pathological characteristics including HER2 status is shown in Table 1. A significant correlation between TOP2A status and several of the clinical and pathological characteristics including age were found. The proportion of women with TOP2A aberrations was increasing with age and as a consequence shown to be higher in the postmenopausal than in the premenopausal women. Further, the proportion of women with TOP2A aberrations increases with tumor size and number of positive lymph nodes. TOP2A aberrations were also found more frequently among the hormone receptor negative or unknown tumors than among the hormone receptor positive tumors. The associations between TOP2A aberrations and age at surgery, tumor size and number of positive lymph nodes are illustrated in FIGS. 2-4.

The distribution of TOP2A aberrations in relation to the HercepTest score and HER2 amplification is shown in Table 2 and 3. In both cases a significant correlation between the TOP2A and the HER2 test result were found (χ2-test; p<0.0001). The distribution of TOP2A amplifications and deletions in relation to the HER2 status is shown in Table 4. As for the HercepTest and HER2 FISH data, not surprisingly, a significant correlation was found between TOP2A status and HER2 status (χ2-test; p<0.0001), with more TOP2A aberrations among the HER2 positive tumors. TOP2A aberrations were seen in 139 (56.5%) of the 246 HER2 positive tumors and 40 (7.6%) of the 527 HER2 negative tumors.

The Kaplan-Meier estimator plots for the survival functions, RFS and OS, with respect to the TOP2A aberrations and HER2 status are shown in FIGS. 5a, 5b, 6a and 6b and summarized in Tables 2-4. Table 1 shows Clinical and pathological characteristics in relation to TOP2A aberrations (N=773)

TABLE 1

| | | | Deleted | | Normal | | Amplified | | p-value |
|---|---|---|---|---|---|---|---|---|---|
| | | N | N (%) | | N (%) | | N (%) | | χ² |
| Treatment | CEF | 352 | 37 | (10.5) | 269 | (76.4) | 46 | (13.1) | 0.59 |
| | CMF | 421 | 50 | (11.9) | 325 | (77.2) | 46 | (10.9) | |
| Menopause | Pre | 535 | 51 | (9.5) | 433 | (80.9) | 51 | (9.5) | 0.0003 |
| | Post | 238 | 36 | (15.1) | 161 | (67.7) | 41 | (17.2) | |
| Age at surgery (yrs.) | -39 | 127 | 7 | (5.5) | 109 | (85.8) | 11 | (8.7) | 0.0066 |
| | 40-49 | 368 | 43 | (11.7) | 289 | (78.5) | 36 | (9.8) | |
| | 50-59 | 170 | 19 | (11.2) | 126 | (74.1) | 25 | (14.7) | |
| | 60-69 | 108 | 18 | (16.7) | 70 | (64.8) | 20 | (18.5) | |
| Size mm | 0-20 | 316 | 25 | (7.9) | 265 | (83.9) | 26 | (8.2) | 0.003** |
| | 21-50 | 392 | 53 | (13.5) | 285 | (72.7) | 54 | (13.8) | |
| | -51 | 63 | 8 | (12.7) | 43 | (68.3) | 12 | (19.0) | |
| | Unknown | 2 | 1 | (50.0) | 1 | (50.0) | 0 | (0.0) | |
| No. of positive nodes | None | 283 | 16 | (5.7) | 246 | (86.9) | 21 | (7.4) | <0.0001 |
| | 1-3 | 244 | 28 | (11.5) | 181 | (74.2) | 35 | (14.3) | |
| | -4 | 246 | 43 | (17.5) | 167 | (67.9) | 36 | (14.6) | |
| Removed lymph nodes | 0-3 | 9 | 2 | (22.2) | 5 | (55.6) | 2 | (22.2) | 0.97 |
| | 4-9 | 282 | 30 | (10.6) | 220 | (78.0) | 32 | (11.4) | |
| | 10- | 482 | 55 | (11.4) | 369 | (76.6) | 58 | (12.0) | |
| Malignancy grade | I | 53 | 5 | (9.4) | 42 | (79.3) | 6 | (11.3) | 0.83** |
| | II | 361 | 47 | (13.0) | 270 | (74.8) | 44 | (12.2) | |
| | III | 309 | 32 | (10.4) | 240 | (77.7) | 37 | (12.0) | |
| | Non-ductal | 46 | 3 | (6.5) | 38 | (82.6) | 5 | (10.9) | |
| | Unknown | 4 | 0 | (0.0) | 4 | (100.0) | 0 | (0.0) | |
| Hormone Receptor status | Positive | 202 | 14 | (6.9) | 168 | (83.2) | 20 | (9.9) | 0.03 |
| | Negative* | 571 | 73 | (12.8) | 426 | (74.6) | 72 | (12.6) | |
| HER2 status | Negative | 527 | 26 | (4.9) | 487 | (92.4) | 14 | (2.7) | <0.0001 |
| | Positive | 246 | 61 | (24.8) | 107 | (43.5) | 78 | (31.7) | |

*Receptor Negative and unknown
**P-values when the unknown observations are omitted

TABLE 2
Distribution of HercepTest score in relation to TOP2A aberrations (N = 773)

| | | | Deleted | | Normal | | Amplified | | p-value |
|---|---|---|---|---|---|---|---|---|---|
| | TOP2A | N | N (%) | | N (%) | | N (%) | | χ² |
| HercepTest | 0 | 209 | 11 | (5.3) | 192 | (91.9) | 6 | (2.9) | p < 0.0001 |
| | 1+ | 257 | 13 | (5.1) | 238 | (92.6) | 6 | (2.3) | |
| | 2+ | 78 | 3 | (3.9) | 65 | (83.3) | 10 | (12.8) | |
| | 3+ | 229 | 60 | (26.2) | 99 | (43.3) | 70 | (30.6) | |
| | Total | 773 | 87 | (11.3) | 594 | (76.8) | 92 | (11.9) | |

TABLE 3
Distribution HER2 FISH ratio in relation to TOP2A aberrations (N = 773)

| | | | Deleted | Normal | Amplified | p-value |
|---|---|---|---|---|---|---|
| | TOP2A | N | N (%) | N (%) | N (%) | χ² |
| HER2 FISH | Normal | 539 | 31 (5.8) | 493 (91.5) | 15 (2.8) | p < 0.0001 |
| | Amplified | 234 | 56 (23.9) | 101 (43.2) | 77 (32.9) | |
| | Total | 773 | 87 (11.3) | 594 (76.8) | 92 (11.9) | |

TABLE 4
Distribution of HER2 status in relation to TOP2A aberrations (N=773)

| | | | Deleted | Normal | Amplified | p-value |
|---|---|---|---|---|---|---|
| | TOP2A | N | N (%) | N (%) | N (%) | χ² |
| HER2 Status | Negative | 527 | 26 (4.9) | 487 (92.4) | 14 (2.7) | p < 0.0001 |
| | Positive | 246 | 61 (24.8) | 107 (43.5) | 78 (31.7) | |
| | Total | 773 | 87 (11.3) | 594 (76.8) | 92 (11.9) | |

Both for RFS and OS, the log-rank test showed association between TOP2A aberrations and survival. Patients with amplifications and deletions had a significant (p<0.0001) reduction in survival compared to patients with a normal TOP2A status. The survival curves seem to indicate that patients with deletions had an even worse prognosis than patients with an amplified or normal TOP2A tumor. Similar, a positive HER2 status was associated with a statistically significant reduction in survival both for RFS and OS (p<0.0001) compared to patients with a negative HER2 status.

The basic Cox model was adjusted for treatment, menopausal status, tumor size, number of positive lymph nodes, HER2 positivity and TOP2A status. Furthermore interaction terms between TOP2A status and treatment and HER2 status respectively were included in the model. As described previously hormone receptor-status and malignancy grade were found to violate the assumption of proportional hazards, and the model was stratified according to these two variables. This model was used in both the analyses of RFS and OS and carried out on the basis of the population of 767 patients. For RFS, it was shown that patients with TOP2A gene aberrations had a significant worse prognosis compared to the TOP2A normals (deletion Hazard Ratio (HR)=1.43, 95% Confidence Interval (CI) 0.80-2.57; amplification HR=2.69, 95% CI 1.18-6.14, p=0.0357). The results for OS showed a similar significant association (deletion HR=1.98, 95% CI 1.09-3.57; amplification HR=2.40, 95% CI 1.05-5.52, p=0.0124). The HR and the 95% confidence limits based on Cox model for RFS and OS are shown in Table 5 and 6. The similar analysis for the HER2 status showed only to be significant with respect to OS, (HER2+HR=1.44, 95% CI 1.06-1.95, p=0.0212).

TABLE 5
HR for RFS-TOP2A and HER2 interaction included (n = 767)

| Variable | p-value | HR | 95% Cl |
|---|---|---|---|
| Menopause status | 0.0461 | | |
| Pre | | 1 | |
| Post | | 1.28 | (1.00-1.62) |
| Tumour size | <0.0001 | | |
| pr. increasing cm | | 1.15 | (1.08-1.22) |
| Positive lymph nodes | <0.0001 | | |
| 0 | | 1 | |
| 1-3 | | 2.06 | (1.42-2.99) |
| 4- | | 4.25 | (2.93-6.16) |
| Treatment | 0.6242 | | |
| CMF | | 1 | |
| CEF | | 0.94 | (0.74-1.20) |
| TOP2A status | 0.0357 | | |
| Deleted | | 1.43 | (0.80-2.57) |
| Normal | | 1 | |
| Amplified | | 2.69 | (1.18-6.14) |
| HER2 status | 0.3236 | | |
| Negative | | 1 | |
| Positive | | 1.16 | (0.86-1.57) |
| Interaction | 0.0091 | | |
| TOP2A × treatment | | | |
| Deleted*CEF | | 0.63 | (0.34-1.17) |
| Amplified*CEF | | 0.38 | (0.20-0.73) |
| Interaction | 0.2737 | | |
| TOP2A × HER2 | | | |
| Deleted*positive | | 1.22 | (0.62-2.41) |
| Amplified*positive | | 0.55 | (0.23-1.27) |

TABLE 6
HR for OS-TOP2A and HER2 interaction included (n = 767)

| Variable | P-value | HR | 95% Cl |
|---|---|---|---|
| Menopause status | 0.0104 | | |
| Pre | | 1 | |
| Post | | 1.38 | (1.08-1.77) |
| Tumour size | <0.0001 | | |
| pr. increasing cm | | 1.16 | (1.09-1.23) |
| Positive lymph nodes | <0.0001 | | |
| 0 | | 1 | |
| 1-3 | | 2.66 | (1.73-4.10) |
| 4- | | 5.55 | (3.61-8.52) |
| Treatment | 0.4412 | | |
| CMF | | 1 | |
| CEF | | 0.90 | (0.69-1.18) |
| TOP2A status | 0.0124 | | |
| Deleted | | 1.98 | (1.09-3.57) |
| Normal | | 1 | |
| Amplified | | 2.40 | (1.05-5.52) |
| HER2 status | 0.0212 | | |
| Negative | | 1 | |
| Positive | | 1.44 | (1.06-1.95) |
| Interaction | 0.0989 | | |
| TOP2A × treatment | | | |
| Deleted*CEF | | 0.75 | (0.4-1.37) |
| Amplified*CEF | | 0.50 | (0.26-0.96) |
| Interaction | 0.2859 | | |
| TOP2A × HER2 | | | |
| Deleted*positive | | 0.85 | (0.43-1.67) |
| Amplified*positive | | 0.51 | (0.22-1.19) |

### Discussion

A number of studies have shown a relationship between TOP2A gene aberration and sensitivity to anthracyclin based chemotherapy [Harris et al., op cit.; Di Leo et al., op cit.; Park et al., op cit.; Press et al., op cit.; Tanner et al., op cit., Knoop et al., op cit.] but so far only one study has looked at the prognostic properties of TOP2A amplification [Callagy et al., op cit.]. The present inventive study is the first to investigate TOP2A gene aberrations (deletion and amplification) in relation to the prognosis of breast cancer. The results of the present inventive study show that the TOP2A gene aberrations are significantly associated with several of the established prognostic factors, such as lymph node status, tumor size, age, ER/PR receptor status and HER2 status. Further, the data of, the present inventive study demonstrate that the proportion of patients with TOP2A aberrations was increasing with age resulting in a higher frequency among postmenopausal than premenopausal patients. Besides the association with the established clinical prognostic factors, it is herein shown that TOP2A aberrations have an independent prognostic value. Using the Cox proportional hazard model, the present inventors have found that a TOP2A gene aberration is associated with a significantly worse prognosis, both with respect to RFS (p=0.04) and OS (p=0.01). The inventors have found no significant interaction between TOP2A aberrations and HER2 status using the Cox model, which emphasizes the independent prognostic value of TOP2A. The univariate survival analyses of the present work illustrates a negative significant effect on both RFS and OS, as patients with amplifications and deletions had a significant reduction in survival compared to patients with a normal TOP2A status. The survival curves also indicate that patients with deletions had an even worse prognosis than patients with an amplified or normal TOP2A status.

When the different prognostic variables in the DBCG 89-D study were ranked based on the HRs for RFS, the ranking was as following: number of positive lymph nodes >TOP2A status>menopausal status>tumor size>HER2 status. The ranking showed that the number of positive lymph nodes was the one variable that had the greatest prognostic impact, which is well established knowledge [Goldhirsch A, Glick J H, Gelber R D, Senn H J. “Meeting highlights: International Consensus Panel on the Treatment of Primary Breast Cancer.” J Natl Cancer Inst 1998; 90(21):1601-8]. It was somewhat more surprisingly to learn that the TOP2A status came out second. However, this ranking should be interpreted with caution due to the interactions with other prognostic factors and the size of the 95% confidence intervals for HRs. Using the topoisomerase IIα overexpression, Rudolph et al showed a similar ranking sequence in a retrospective study of tumour tissue from 863 patients with node negative breast cancer [Rudolph, MacGrogan et al., op cit]. If the ranking in DBCG 89-D study is repeated for OS, the results are similar as for RFS.

Based on both univariate and multivariate analyses, the results from the DBCG 89-D study demonstrate significant prognostic value of TOP2A aberrations. Not only are the TOP2A aberrations associated with the already-established prognostics factors in breast cancer, but are also shown to possess an independent prognostic value. Predetermined Kaplan-Meier estimator plots for the survival functions, RFS and OS, such as those shown in FIGS. 5a, 5b, may be used to perform prognoses for individual breast cancer patients according to some methods in accordance with the present invention. First, the status of an aberration of the TOP2A gene in a tissue sample taken from the patient is determined as described above. This may include performing TOP2A FISH analysis on the tissue sample, for instance using the TOP2A FISH pharmDx™ Kit noted above. Once the correct TOP2A aberration status (normal, amplification or deletion) has been determined, the appropriate curve (corresponding to the determined status) on the Kaplan-Meier estimator plot is consulted in order to estimate the probability of either recurrence-free survival or of overall survival of the patient at a later time. Alternatively, a pre-determined Hazard Ratio corresponding to the determined status, such as those provided in the Tables 5-6, may be used in the calculation of such probabilities. Alternatively, a clinician may use the data provided herein as a basis for making his/her own professional prognostic evaluation based on the marker status.

Novel methods for performing prognoses for high-risk breast cancer patients using TOP2A gene aberrations has been disclosed. The essence of the invention includes not only the disclosed methods but, also, the various systems, assemblies, and devices required or usable to accomplish these methods. Those skilled in the art can now appreciate, from the foregoing description, that the broad techniques of the embodiments of the present invention can be implemented in a variety of forms. Therefore, although the invention has been described in connection with specific examples or embodiments, it should be understood that the invention as claimed is not intended to be and should not be unduly limited to such specific examples or embodiments. Indeed, various modifications of the described modes for carrying out the invention which are obvious to those skilled in diagnostic pathology or related fields are intended to be within the scope of the claims.

### Example 2

We have analyzed the dataset of 773 patients. The Kaplan-Meier survival analysis was conducted to the end of follow-up time. There were 352 CEF (Cyclophosphamide-Epirubicin-5-Fluorouracil treatment) patients and 421 CMF (Cyclophosphamide-Methotrexate-5-Fluorouracil) patients. Among 352 CEF patients, there were 269 TOP2A normals, 46 TOP2A amplifications and 37 TOP2A deletions. We have found that, within CEF, there was no significant difference among the three subgroups (TOP2A amplification, normal, deletion) for recurrence free survival (p=0.1423) but there was statistical significance for overall survival (p=0.0022). Submit the Kaplan-Meier survival plots with 767 patients for each treatment separately, one plot for CEF and another for CMF. Each plot should show the three different subgroups.

### Report P-Values and Discussion

In the data material cointaining 767 patients univariate analyses for each treatment (CEF and CMF) are performed using Kaplan-Meier survival plots (KM-plots). The curves are stratified according to the three groups of TOP2A status, and in order to test homogeneity of the survival curves across strata a Log-rank test is calculated. Furthermore, a Log-rang test comparing normal with amplified and deleted tumors, respectively, are carried out.

### KM-Plot for RFS

Among patients treated with CMF the KM-plot indicates a significant difference between the three groups of the TOP2A status (FIG. 7). The p-value from the Log-rank test is p<0.0001.

In the group of patients treated with CEF this difference is not retrieved, the p-value in this group is p=0.1386 (FIG. 8).

The table below shows the p-values from the Log-rank tests when comparing normal with deleted and amplified separately for each of the Kaplan-Meier curves.

Table 7 below demonstrates p-values for RFS when comparing strata pair-wise.

TABLE 7

| Treatment | Strata compared | p-value |
|---|---|---|
| CMF | Deleted vs. normal | <0.0001 |
| | Amplified vs. normal | 0.0058 |
| CEF | Deleted vs. normal | 0.0505 |
| | Amplified vs. normal | 0.9763 |

Table 8 below shows the number of patients at risk, RFS with 95% confidence limits at the two time points 5 and 10 years after randomization for each group of TOP2A and divided by treatment.

TABLE 8

| | | CMF | | | CEF | | |
|---|---|---|---|---|---|---|---|
| TOP2A | Time | No. at | Survival | | No. at | Survival | |
| group | (years) | risk | (%) | 95% Cl | risk | % | 95% Cl |
| Deleted | 5 | 14 | 28.6 | (15.9-41.2) | 14 | 47.7 | (30.8-64.6) |
| | 10 | 3 | 25.7 | (13.2-38.3) | 3 | 40.6 | (23.6-57.6) |
| Normal | 5 | 181 | 61.2 | (55.8-66.6) | 158 | 64.4 | (58.5-70.3) |
| | 10 | 42 | 51.2 | (45.4-56.9) | 35 | 52.1 | (45.4-58.7) |
| Amplified 10 | 5 | 17 | 41.0 | (26.4-55.6) | 23 | 57.8 | (42.9-72.7) |
| | 10 | 2 | 31.0 | (16.1-45.8) | 6 | 55.3 | (40.2-70.3) |

The recurrence free survival is improved for patients having TOP2A amplified or deleted tumors treated with CEF, in the way that RFS for these groups of patients is not different from the TOP2A normal.

### KM-Plot for OS

When looking at overall survival, again we find a difference in TOP2A status with a p-value of p<0.0001 among patients treated with CMF. The KM-plot are shown in FIG. 9 In contrast to RFS this difference is still significant in the group of patients treated with CEF (FIG. 10). The p-value from the Log-rank test is p=0.0024. The p-values for OS from the pair-wise comparisons are shown in Table 9 below.

TABLE 8

| Treatment | Strata compared | p-value |
|---|---|---|
| CMF | Deleted vs. normal | <0.0001 |
| | Amplified vs. normal | 0.0069 |
| CEF | Deleted vs. normal | 0.0008 |
| | Amplified vs. normal | 0.1721 |

It is only in the TOP2A amplified case that treatment with CEF brings OS to the same level as for the TOP2A normal. But the relative improvement in OS for the deleted patients is not less pronounced.

Table 10 shows number of patients at risk and survival at 5 and 10 years for each TOP2A group by treatment arm.

TABLE 10
OS at 5 and 10 yrs. in each TOP2A group by treatment

| | | CMF | | | CEF | | |
|---|---|---|---|---|---|---|---|
| TOP2A | Time | No. at | Survival | | No. at | Survival | |
| group | (years) | risk | (%) | 95% Cl | risk | % | 95% Cl |
| Deleted | 5 | 17 | 34.7 | (21.4-48.0) | 19 | 51.4 | (35.2-67.5) |
| | 10 | 9 | 26.1 | (13.6-38.5) | 9 | 43.2 | (27.3-59.2) |
| Normal | 5 | 233 | 72.1 | (67.2-77.0) | 200 | 75.2 | (70.0-80.4) |
| | 10 | 127 | 60.0 | (54.5-65.5) | 112 | 66.4 | (60.6-72.2) |
| Amplified | 5 | 22 | 47.8 | (33.4-62.3) | 31 | 67.4 | (53.8-80.9) |
| | 10 | 11 | 43.3 | (28.9-57.6) | 15 | 53.5 | (38.8-68.3) |

### SUMMARY AND CONCLUSION

In the group of patients treated with CEF the Kaplan-Meier plots for recurrence free survival (RFS) show no difference between the patients with TOP2A deleted, normal or amplified tumors (p=0.1386). With respect to overall survival (OS), for the same group of patients, a significant difference between the three TOP2A groups is seen (p=0.0024). In the CMF arm, there is a significant difference between the TOP2A groups for both RFS and OS (p<0.0001), where patients with normal TOP2A gene status seem to have the best survival.

The Kaplan-Meier plots shows that treatment with CEF improves RFS and OS for patients with both deleted and amplified tumors. For patients with TOP2A amplified tumors, treatment with CEF brings RFS and OS to the same level as for the TOP2A normal. Patients with TOP2A deleted tumors have the same relative improvement in RFS and OS as amplified patients, but they do not seem to reach the same level of survival as the patients with TOP2A amplified and normal tumors.

1. A method for performing a prognosis for a breast cancer patient, comprising the steps: determining the status of an aberration of the TOP2A gene in a tissue sample taken from the patient; and estimating the probability of either recurrence-free survival or of overall survival of the patient at a later time based upon a pre-determined Hazard Ratio corresponding to the determined status.

2. The method of claim 1, wherein the determined status corresponds to TOP2A amplification.

3. The method of claim 1, wherein the determined status corresponds to TOP2A deletion.

4. The method of claim 1, wherein the determined status corresponds to normal TOP2A.

5. The method of claim 1, wherein the step of determining the status of an aberration includes conducting a Flourescent In-Situ Hybridization (FISH) analysis of the tissue sample.

6. The method of claim 5, wherein the conducting a Flourescent In-Situ Hybridization (FISH) analysis of the tissue sample comprises using a probe mixture comprising Texas Red-labelled DNA probes targeted at a portion of the TOP2A region and a probe mixture comprising fluorescein-labelled Peptide Nucleic Acid (PNA) probes targeted at the centromeric region of chromosome 17.

7. The method of claim 1, wherein the pre-determined Hazard Ratio is calculated by performing steps comprising: determining the status of aberrations of the TOP2A gene in a set of tissue samples taken from respective patients; and performing subsequent follow-up studies of recurrence-free survival time or of overall survival time for the patients.

8. A method for performing a prognosis for a breast cancer patient, comprising the steps: determining the status of an aberration of the TOP2A gene in a tissue sample taken from the patient; and estimating the probability of either recurrence-free survival or of overall survival of the patient at a later time based upon a pre-determined Kaplan-Meier plot corresponding to the determined status.

9. The method of claim 8, wherein the determined status corresponds to TOP2A amplification.

10. The method of claim 8, wherein the determined status corresponds to TOP2A deletion.

11. The method of claim 8, wherein the determined status corresponds to normal TOP2A.

12. The method of claim 8, wherein the step of determining the status of an aberration includes conducting a Fluorescent In-Situ Hybridization (FISH) analysis of the tissue sample.

13. The method of claim 12, wherein the conducting a Flourescent In-Situ Hybridization (FISH) analysis of the tissue sample comprises using a probe mixture comprising Texas Red-labeled DNA probes targeted at a portion of the TOP2A region and a mixture of fluorescein-labeled Peptide Nucleic Acid (PNA) probes targeted at the centromeric region of chromosome 17.

14. The method of claim 8, wherein the pre-determined Kaplan-Meier plot is calculated by performing steps comprising: determining the status of aberrations of the TOP2A gene in a set of tissue samples taken from respective patients; and performing subsequent follow-up studies of recurrence-free survival time or of overall survival time for the patients.
